(19) United States
(12) Patent Application Publication
(10) Pub. No.: US 2012/0245463 A1
(43) Pub. Date: Sep. 27, 2012

(54) TOMOSYNTHESIS APPARATUS AND METHOD

(76) Inventor: Thomas Mertelmeier, Erlangen (DE)

(21) Appl. No.: 13/428,194

(22) Filed: Mar. 23, 2012

(30) Foreign Application Priority Data
Mar. 24, 2011 (DE) 10 2011 006 058.8

(51) Int. Cl. A61B 6/02; A61B 8/13

(52) U.S. Cl. 600/427

(57) ABSTRACT

A tomosynthesis apparatus has an x-ray source to generate an x-ray beam directed toward an x-ray detector and a support plate and a compression plate between which a breast can be positioned. The compression plate has a compression surface that is concave toward the support plate, and is provided with at least one through-hole into which an ultrasound transducer is inserted for a sonographic data acquisition.

### BACKGROUND OF THE INVENTION

1. Field of the Invention

The invention concerns a tomosynthesis apparatus and a method for its operation of a tomosynthesis apparatus.

2. Description of the Prior Art

Tomosynthesis is a development for x-ray examination of the breast that is within the category of x-ray mammography, with which it is possible to generate a three-dimensional image data set of slice images in order to be able to detect malignant lesions at an earlier stage than in conventional x-ray mammography, and thereby to improve the prospects of recovery. Both conventional x-ray mammography and tomosynthesis lose sensitivity in dense tissue, i.e. particularly in young women, such that fewer lesions are discovered with increasing density of the breast. While the sensitivity of x-ray mammography in women over 50 is between 70 and 95%, for instance, in young women with dense breast tissue it falls below 50%. Given the presence of a dense breast tissue, it is known to implement a sonographic examination as an alternative or in addition to x-ray mammography. With such a sonographic examination, lesions or malignant tumors can be discovered that would remain undetected with x-ray mammography.

Since a conventional, manually directed ultrasound examination takes place differently (i.e. on an uncompressed breast) than x-ray mammography or tomosynthesis, making a spatial association of subjects in images of the two modalities is difficult. In order to be able to enable such an association, for example, it is known from U.S. Pat. Nos. 6,574,499 and 6,876,879 to implement an ultrasound examination while the breast is located in a compressed state, as is the case in x-ray mammography. For this purpose, an ultrasound transducer is set up on the flat compression plate of a conventional mammography apparatus and directed over this in order to generate a three-dimensional ultrasound image from the echo signals. However, both the relatively long acquisition time (caused by the movement of the ultrasound transducer on the compression plate)—that can be stressful for the examined person due to the compression of the breast—and the fact that the ultrasound must pass through multiple interfaces (boundary layers), both upon injection into the breast and in the decoupling of the echo signal, are problematical.

### SUMMARY OF THE INVENTION

An object of the invention is to provide a tomosynthesis apparatus with which it is possible to generate both x-ray images and ultrasound images, and in which the problems described above are largely avoided. A further object of the invention is to provide a method to operate such a tomosynthesis apparatus.

With regard to the tomosynthesis apparatus, the above object is achieved according to the invention is by a tomosynthesis apparatus wherein the compression plate has a compression surface that is concave toward the support plate and is provided with at least one through-hole in which an ultrasound transducer for a sonographic acquisition is inserted.

By providing a through-hole for the ultrasound transducer, the transducer can be placed directly against the breast via a coupling gel, such that no additional interfaces at which reflections occur (which interferes with the echo signal) are present between transmission and reception surface of the ultrasound transducer, and the surface of the breast.

In order to enable a sonographic imaging of the entire breast, and in particular to enable a three-dimensional ultrasound image of the breast, the at least one ultrasound transducer is advantageously mounted such that it can pivot in the through-hole.

With regard to a method to operate the tomosynthesis apparatus, the above object is achieved according to which at least one radiographic projection image is acquired from which a value for the breast density is determined, and in which a decision as to whether an ultrasound examination is implemented or not is made depending on this value.

In a further embodiment of the method to operate the tomosynthesis apparatus, both a 3D ultrasound image and a tomosynthetic 3D x-ray image are generated, and both images are brought into registration so that they can be combined or superimposed with spatial accuracy.

### BRIEF DESCRIPTION OF THE DRAWINGS

FIG. 1 schematically illustrates a tomosynthesis apparatus according to the invention.

FIGS. 2-4 respectively show alternative embodiments of a compression plate provided for use in a tomosynthesis apparatus according to the invention, each in a plan view.

### DESCRIPTION OF THE PREFERRED EMBODIMENTS

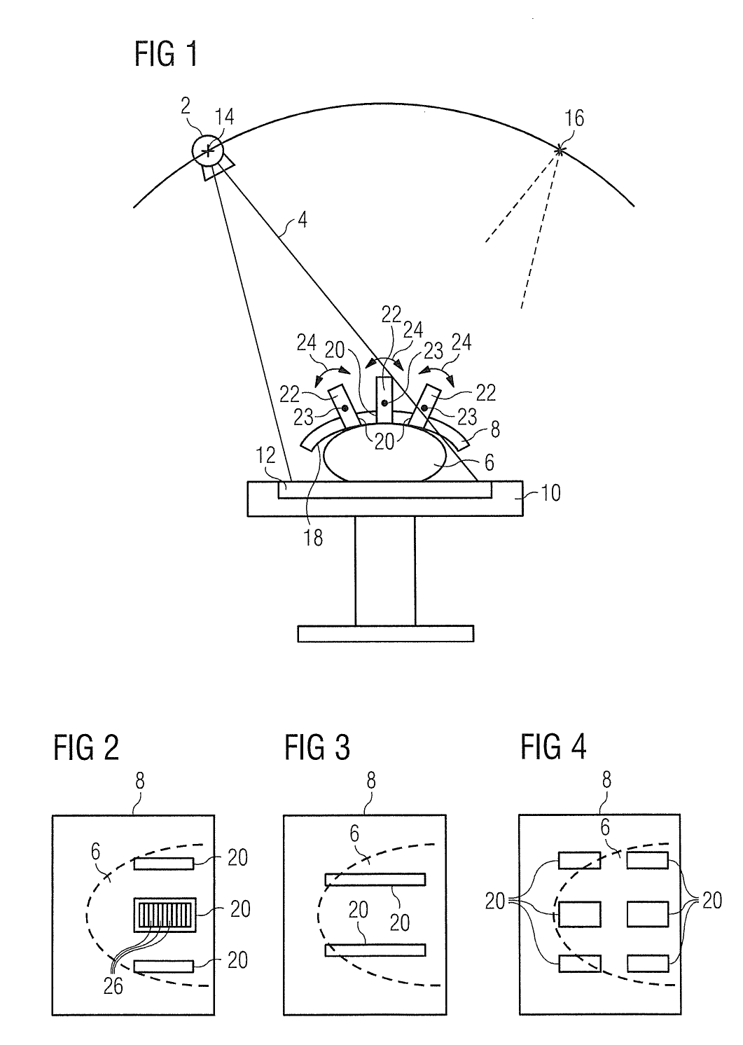

As shown in FIG. 1, a tomosynthesis apparatus according to the invention has an x-ray source 2 (normally an x-ray tube) that emits an x-ray beam 4. The x-ray beam 4 traverses a breast 6 that is positioned and compressed between a compression plate 8 and a support plate 10. An x-ray detector 12—in the exemplary embodiment a digital flat panel detector with which the x-ray beam 4 emitted by the x-ray source 2 is received—is countersunk into the support plate 10.

The x-ray source 2 is supported so as to be spatially movable relative to the support plate 10 in order to generate projection images from different angle directions. In the shown example, the x-ray source 2 is mounted such that it can pan in a limited angle range between two end positions 14, 16. As an alternative to such an x-ray source 2 arranged to pan, an x-ray source can be provided that can be moved along a straight track and can be pivoted on a pivot axis oriented perpendicularly to this linear track. The x-ray source 2 can also be designed as a type known as a linear multifocus x-ray tube, that has individual emitters arranged along a straight line that can be activated independently of one another, such that in this case the focus of the x-ray source is also shifted along a straight line.

The compression plate 8 has a concave compression surface 18 facing toward the support plate 10. Such a concave design of the compression surface 18 is advantageous for tomosynthesis since, as explained in detail in U.S. Pat. No. 7,742,558, a high compression of the breast 6 is not necessary, in contrast to conventional mammography.

The compression plate 8 has a number of through-holes 20 into any of which an ultrasound transducer 22 is inserted that, in practice, is placed directly against the breast 6 (i.e. separated from the skin surface by only a thin intermediate layer composed of a coupling gel, applied before the insertion of the ultrasound transducer 22 into the through-hole 20). Multiple transducers 22 can be simultaneously used. In order to not interfere with the radiographic acquisitions, the ultrasound transducers 22 are not inserted into the through-holes 20 during the radiographic acquisitions.

An ultrasound transducer arrangement in which multiple ultrasound transducers are arranged next to one another in a longitudinal direction in a linear phased array, which ultrasound transducer elements can be activated with a time delay relative to one another, is advantageously provided as an ultrasound transducer 22. The longitudinal direction of the ultrasound transducer arrangement extends perpendicularly to the plane of the drawing, such that slice images of the breast 6 that proceed orthogonally to the plane of the drawing can be generated with the linear array. In order to be able to generate a complete three-dimensional ultrasound image of the entire breast 6, the ultrasound transducers 22 are respectively mounted such that they can pivot around a pivot axis 23 proceeding orthogonally to the plane of the drawing, as is indicated by the double arrows 24. Since the position and compression of the breast 6 remains the same in the generation of a tomosynthetic image data set (3D x-ray image) and in the generation of a 3D ultrasound image data set, both data sets (i.e. 3D ultrasound image and 3D x-ray image) can be superimposed with one another with spatial accuracy, i.e. they can be accurately brought into registration with each other.

Various embodiments of the compression plate 8 that respectively include a different number of through-holes 20 are shown in the plan views according to FIG. 2-4. Moreover, an ultrasound transducer having a number of ultrasound transducer elements that is inserted into the middle through-hole 20 is shown in simplified form in FIG. 2.

Although modifications and changes may be suggested by those skilled in the art, it is the intention of the inventor to embody within the patent warranted hereon all changes and modifications as reasonably and properly come within the scope of his contribution to the art.

I claim as my invention:

1. A tomosynthesis apparatus comprising:
an x-ray source that emits an x-ray beam;
a support plate and a compression plate located in a path of said x-ray beam, said support plate and said compression plate being configured to compress a breast there between;
an x-ray detector located in said path of said x-ray beam following said support plate and said compression plate, said x-ray detector emitting an output signal representing attenuated x-rays incident thereon;
said x-ray source being movable to emit said x-ray beam from a plurality of different directions with respect to said x-ray detector, and said x-ray detector emitting respective signals representing said attenuated x-rays from each of said directions;
said compression plate having a compression surface facing said support plate, said compression surface being concave toward said support plate and having at least one through-hole therein; and
an ultrasound transducer in said at least one through-hole configured to acquire sonographic information from the breast between said compression plate and said support plate.

2. A tomosynthesis apparatus as claimed in claim 1 wherein said ultrasound transducer is mounted in said through-hole allowing pivoting of said ultrasound transducer in said through-hole.

3. A tomosynthesis apparatus as claimed in claim 1 wherein said ultrasound transducer comprises a plurality of transducer elements arranged next to each other.

4. A tomosynthesis apparatus as claimed in claim 3 wherein said plurality of transducer elements are configured to form a linear phased array.

5. A method for operating a tomosynthesis apparatus comprising:
acquiring a plurality of x-ray images respectively from different directions by irradiating a breast, compressed between a compression plate and a support plate, with x-rays emitted from an x-ray source, and detecting x-rays attenuated by the breast with an x-ray detector;
providing an ultrasound transducer in said compression plate, said ultrasound transducer being configured to acquire sonographic information from the breast between said support plate and said compression plate; and
from said x-ray images, making a determination of a density of the breast and, dependent on a value of the density of the breast, operating said ultrasound transducer to acquire said sonographic information.

6. A method as claimed in claim 5 comprising generating a 3D ultrasound image with said ultrasound transducer.

7. A method as claimed in claim 6 comprising generating a 3D x-ray image from said x-ray images, and presenting said 3D ultrasound image and said 3D x-ray image in registration with each other.
